(19) United States
(12) Patent Application Publication
(10) Pub. No.: US 2022/0409365 A1
(43) Pub. Date: Dec. 29, 2022

(54) STENT TO BE INSERTED INTO EUSTACHIAN TUBE FOR TREATMENT OF EUSTACHIAN TUBE DYSFUNCTION

(71) Applicant: J&KYM CO., LTD., Gimhae-si, Gyeongsangnam-do (KR)

(72) Inventor: Ho Sung JOE, Busan (KR)

(21) Appl. No.: 17/638,499

(22) PCT Filed: Sep. 4, 2019
(86) PCT No.: PCT/KR2019/011416

§ 371 Date: Feb. 25, 2022

(30) Foreign Application Priority Data
Sep. 3, 2019 (KR) 10-2019-0108865

(51) Int. Cl. A61F 2/18; A61F 2/86; A61F 11/20; A61L 27/06; A61L 27/54

(52) U.S. Cl. CPC A61F 2/18; A61F 2/86; A61F 11/202; A61L 27/06; A61L 27/54; A61F 2002/183; A61F 2210/0014

(57) ABSTRACT

This application relates to a stent inserted in an eustachian tube for treatment of eustachian tube dysfunction. In one aspect, the stent includes a pressure controller which blocks the eustachian tube and is opened/closed according to a pressure difference between front and rear portions thereof to control a pressure in the eustachian tube. The stent may also include a eustachian tube expansion portion which is connected to the pressure controller and has a hollow portion passed therein to make a fluid move in back and forth directions of the eustachian tube, and is inserted in the eustachian tube to expand the eustachian tube by transforming a shape thereof in a radial direction of the eustachian tube.

### TECHNICAL FIELD

The present disclosure relates to a stent to be inserted in a eustachian tube for treatment of eustachian tube dysfunction, and in particular, to a stent to be inserted in a eustachian tube for treatment of eustachian tube dysfunction such as eustachian tube stenosis or abnormal patency of the eustachian tube.

### BACKGROUND ART

A eustachian tube is also referred to as an auditory tube which is a tube having a length of 3 cm to 4 cm and starts from under an internal space of a middle ear and extends to nasopharynx behind the nasal cavity. An upper side of the tube is surrounded by a narrow bone and a lumen of the tube increases to change to a cartilage bone in a downward direction. There are small cilia in the tube to help secreta from the middle ear to be discharged to the nasopharynx. Because a eustachian tube of a child is horizontal and has a small length, it is easy for bacteria or viruses to infiltrate into the middle ear and cause infection. It is known that according to the human growth, the eustachian tube has increased in length and is inclined such that mucus and foreign substances may be easily discharged, leading to a decrease in the risk of infection.

Functions of the eustachian tube will be described in detail below with reference to FIGS. 1A and 1B.

A pressure between the tympanic cavity and outside is adjusted by opening/closing the eustachian tube connecting an inside of an ear drum (tympanic cavity) to a side of nasopharynx (nasal cavity, pharyngeal cavity). As shown in of FIG. 1A, when the eustachian tube is blocked, the atmospheric pressure out of the ear drum is relatively greater than the pressure in the middle ear, the ear drum is stretched inward to the side of the tympanic cavity according to a pressure difference. On the contrary, as shown in FIG. 1B, when the eustachian tube is opened, the pressure in the middle ear and the atmospheric pressure outside the ear drum are balanced.

The eustachian tube is normally closed, and then, is opened when a person swallows saliva or yawns such that the pressure in the middle ear is balanced with the atmospheric pressure. In particular, a person feels pain in the ear during a flight because the eustachian tube does not operate right (does not open), and then a balanced state of the ear drum is broken due to the pressure difference between an external acoustic meatus and the tympanic cavity based on the ear drum. A status in which the eustachian tube's performance of ventilating the middle ear and adjusting the pressure degrades is referred to as ‘eustachian tube dysfunction’, and representative examples of the eustachian tube dysfunction include abnormal patency of the eustachian tube and eustachian tube stenosis.

A symptom of continuously maintaining the opening state of the eustachian tube is referred to as abnormal patency of the eustachian tube. Although a degree of pain is very subjective to each individual, the abnormal patency of the eustachian tube is known as largely affecting the everyday life. Main symptoms include aural fullness (deafening feeling), movement of ear drum felt in breathing, self-voice listening (one's own voice being too loud), etc. From among the above symptoms, the self-voice listening denotes a phenomenon in which echo is too loud to control one's own voice, which causes discomfort during conversation, and even breathing sound is too loud. Known causes of the abnormal patency of the eustachian tube include weight loss, issues regarding hormones, allergic rhinitis, reflux esophagitis, and issues regarding mucus in the eustachian tube, etc.

In addition, a symptom of continuously closing the eustachian tube is referred to as the eustachian tube stenosis. The eustachian tube stenosis makes the eustachian tube continuously closed, which results in thin air in the middle ear space. Therefore, a patient experiences deafening feeling as if he/she was in a flight, and the ear drum is retracted inward. Then, one's own voice echoes, and moreover, stuffy and deafening feeling become stronger. Thus, may be a slight hearing loss.

There is no researched cure, rather than natural healing, for the eustachian tube dysfunction as described above, actions such as balsalva (blocking nose and exhaling) or sniffing are mainly performed, but the above actions may cause tympanic membrane retraction, etc. and should be avoided.

The above eustachian tube dysfunction may cause barotitis and otitis media with effusion.

Atmospheric pressure in the external acoustic meatus gradually increases when an airplane descends to a destination. On the contrary, an air pressure in the tympanic cavity inside the ear drum is relatively low. When the performance of the eustachian tube normally operates, the eustachian tube is naturally opened due to a swallowing action. Then, the pressures of the external acoustic meatus and the tympanic cavity are instantly balanced, and the ear drum maintains its own position. Thus, the deafening feeling disappears and there is no specific symptom. However, when a person has the eustachian tube dysfunction, the pressure in the tympanic cavity is relatively negative as compared with the external acoustic meatus, a volume decreases, and accordingly, the tympanic membrane retraction is caused and pain in the ear occurs. As described above, otitis media caused by rapid change in the peripheral pressure is referred to as ‘barotitis’, and those who work as divers or do scuba diving, as well as the flight, may frequently experience such pain under the deep sea water.

Also, when the barotitis is remained untreated and frequently recurs, the negative pressure state of the tympanic cavity is continued, which causes swelling of mucous membrane in the middle ear and expansion of capillary. Then, the barotitis may be developed to the otitis media with effusion, by which effusion fluid pools in the tympanic cavity. In addition, in case of the eustachian tube stenosis, the eustachian tube is blocked and infection materials accumulated in the middle ear may not be discharged, and then, the eustachian tube stenosis mostly becomes the otitis media with effusion.

Treatments for eustachitis may be classified as surgical treatments and non-surgical treatments. The non-surgical treatment includes two to three weeks of medication. Infections generated on mucous membrane of the eustachian tube are removed through administration of anti-inflammatory drugs, anti-biotics, etc. When the symptoms persist even with the administration, a surgical procedure is necessary.

The surgical treatment is performed when the chronic eustachitis occurs due to structural issues of the eustachian tube, and a ventilation tube insertion, in which a small hole is perforated in the ear drum to ventilate and the infection materials in the middle ear are discharged, is known.

Even when suppuration and infection materials are removed by performing the ventilation tube insertion, problems may occur again after a certain period of time. That is, without addressing the tubal stenosis, the ventilation tube insertion in the middle ear only shows a temporary effect, and there is a room for problems again at any time. Therefore, it is necessary to address the problematic eustachian tube dysfunction caused by the muscle tissue of the eustachian tube not working properly.

### DESCRIPTION OF EMBODIMENTS

### Technical Problem

Provided is a stent to be inserted in a eustachian tube for treatment of eustachian tube dysfunction, the stent is inserted in the eustachian tube to extend the eustachian tube through a shape transformation in a radial direction of the eustachian tube and is opened/closed due to a pressure difference between inside/outside to control a pressure in the eustachian tube, and thus, eustachian tube stenosis and abnormal patency of the eustachian tube may be permanently treated.

It will be appreciated by one of ordinary skill in the art that that the objectives and effects that could be achieved with the inventive concept are not limited to what has been particularly described above and other objectives of the inventive concept will be more clearly understood from the following detailed description.

### Solution to Problem

According to the present disclosure, a stent to be inserted in a eustachian tube for treatment of eustachian tube dysfunction, includes: a pressure controller which blocks the eustachian tube and is opened/closed according to a pressure difference between front and rear portions thereof to control a pressure in the eustachian tube; and a eustachian tube expansion portion which is connected to the pressure controller and has a hollow portion passed therein to make a fluid move in back and forth directions of the eustachian tube, and is inserted in the eustachian tube to expand the eustachian tube by transforming a shape thereof in a radial direction of the eustachian tube.

Here, the eustachian tube expansion portion may be connected to one side or opposite sides of the pressure controller.

Here, the eustachian tube expansion portion may include a body portion which forms a ring shape while extending in a circumferential direction as a pattern in which a waveform of a wire is repeated in the back-and-forth directions of the eustachian tube.

Here, the stent may further include a plurality of connecting frames connecting one side of the eustachian tube expansion portion to one side of the pressure controller.

Here, the eustachian tube expansion portion may further include a plurality of body portions arranged in the back and forth directions of the eustachian tube, and a connecting portion connecting the body portions.

Here, the connection portion may be formed as a wire.

Here, the connection portion may be curved as an S-shape.

Here, the eustachian tube expansion portion may include: a body portion which forms a ring shape while extending in a circumferential direction of the eustachian tube as a pattern in which a waveform is repeated in the circumferential direction; and a connecting portion for connecting a plurality of body portions arranged in back and forth directions of the eustachian tube or the body portion and the pressure controller to each other.

Here, the eustachian tube expansion portion may be formed of a shape memory alloy and thermally treated to memorize a shape that is transformed in the radial direction of the eustachian tube.

Here, the eustachian tube expansion portion may include nitinol (Ni—Ti).

Here, a biocompatible material for preventing bacterial proliferation may be coated on a surface of the stent.

Here, a biodegradable polymer layer may be coated on a surface of the stent, and a drug may be absorbed by the biodegradable polymer layer and is discharged into the eustachian tube.

Here, the pressure controller may include: a fixed portion formed to have a cylindrical shape, in which a hollow portion penetrating therein is formed, to be inserted and support the eustachian tube; and a rotating portion which shields a surface of the hollow portion in the fixed portion and of which an end portion of a side surface in a radial direction thereof is fixed to an inner surface of the fixed portion, the rotating portion rotating due to a pressure difference between front and rear portions thereof to open/close the surface of the hollow portion.

Here, the rotating portion may include a shielding portion covering the surface of the hollow portion in the fixed portion; and a protruding coupling portion which protrudes from an end portion of a side surface in a radial direction of the shielding portion, and a coupling groove portion in which the protruding coupling portion is inserted is formed in the inner surface of the fixed portion.

Here, in the shielding portion, a side surface opposite to the protruding coupling portion in a radial direction may have an inclined surface that is inclined toward a front surface or a rear surface and may form a contact surface that is in contact with the inclined surface and the fixed portion.

### Advantageous Effects of Disclosure

According to a stent to be inserted in a eustachian tube for treatment of eustachian tube dysfunction of the present disclosure, a stent is inserted in the eustachian tube and treats the eustachian tube dysfunction.

Also, the eustachian tube dysfunction is addressed, and thus, a fundamental cause of otitis media with effusion may be addressed.

Also, because the eustachian tube expansion portion has flexibility due to its shape characteristics, the stent formed of metal may transform its shape according to a curved portion in the eustachian tube, and the stent may be stably fixed in the eustachian tube while extending the eustachian tube.

Also, a biocompatible material may be coated on an outer side such that the stent may be permanently inserted in the eustachian tube.

Also, a biodegradable polymer layer may be coated to supply a drug for curing or preventing infections into the eustachian tube via the stent.

### BRIEF DESCRIPTION OF DRAWINGS

FIGS. 1A and 1B are diagrams for describing functions of a eustachian tube.

FIG. 2 is a perspective view of a stent according to an embodiment of the present disclosure.

FIG. 3 is a perspective view showing a shape-memory state of FIG. 2.

FIG. 4 is a perspective view of a pressure controller according to an embodiment of the present disclosure.

FIG. 5 is a cross-sectional view of FIG. 4.

FIG. 6 is a front view of a rotating portion in FIG. 5.

FIG. 7 is a side view of FIG. 6.

FIGS. 8A and 8B are diagrams for illustrating operations of a pressure controller according to a pressure change in a eustachian tube.

FIG. 9 is a perspective view of a stent of FIG. 2, according to a modified example.

FIG. 10 is a perspective view of a stent according to another embodiment of the present disclosure.

FIG. 11 is a perspective view of a stent according to another embodiment of the present disclosure.

FIG. 12 is a diagram showing an example of inserting a stent in a eustachian tube for treating eustachian tube dysfunction according to the present disclosure.

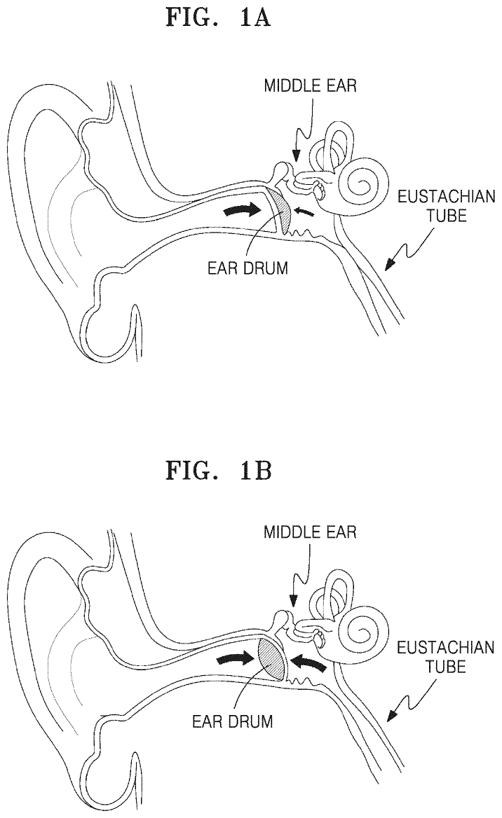

### MODE OF DISCLOSURE

Details of embodiments are included in the following description and the accompanying drawings.

The attached drawings illustrate one or more embodiments and are referred to in order to gain a sufficient understanding, the merits thereof, and the objectives accomplished by the implementation. However, the present disclosure is not limited to the exemplary embodiments set forth below, and may be embodied in various other forms. The embodiments are for rendering the description of the present disclosure complete and are set forth to provide a complete understanding of the scope of the disclosure to one of ordinary skill in the art to which the present disclosure pertains. Throughout the specification, like reference numerals denote the same elements.

Hereinafter, the present disclosure will be described below with reference to accompanying drawings for describing a stent to be inserted in a eustachian tube for treatment of eustachian tube dysfunction according to the embodiments of the present disclosure.

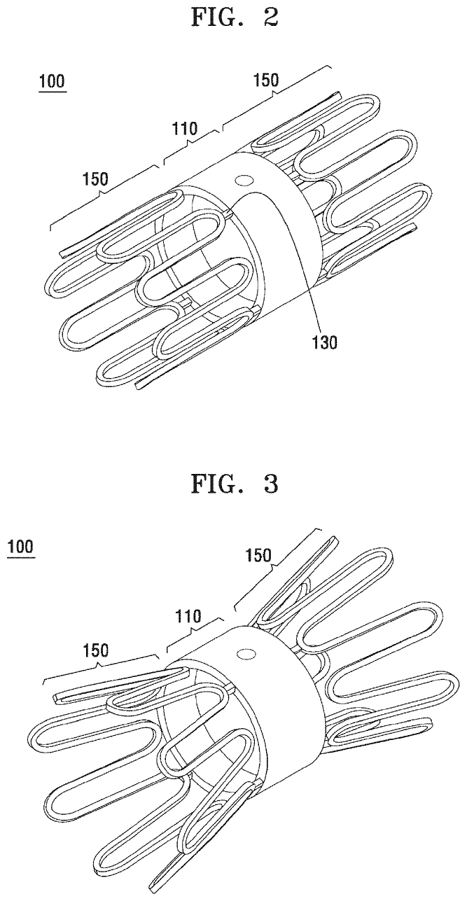

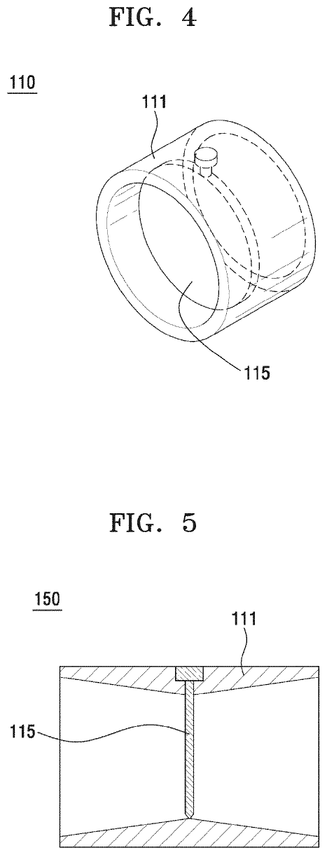

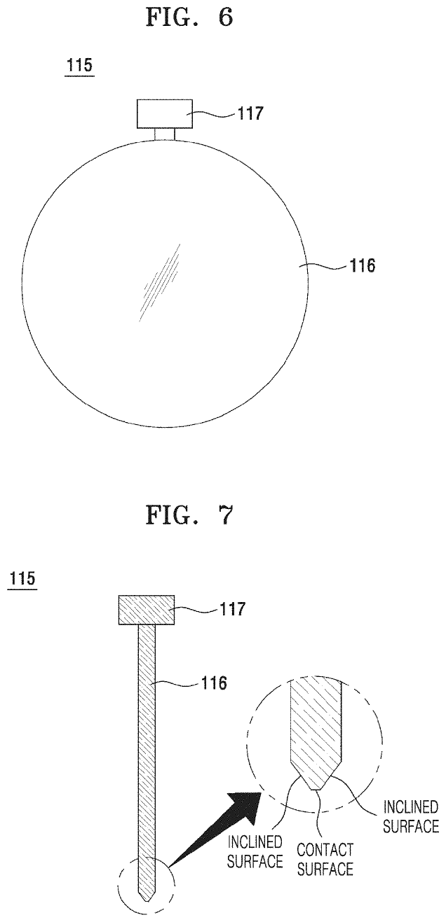

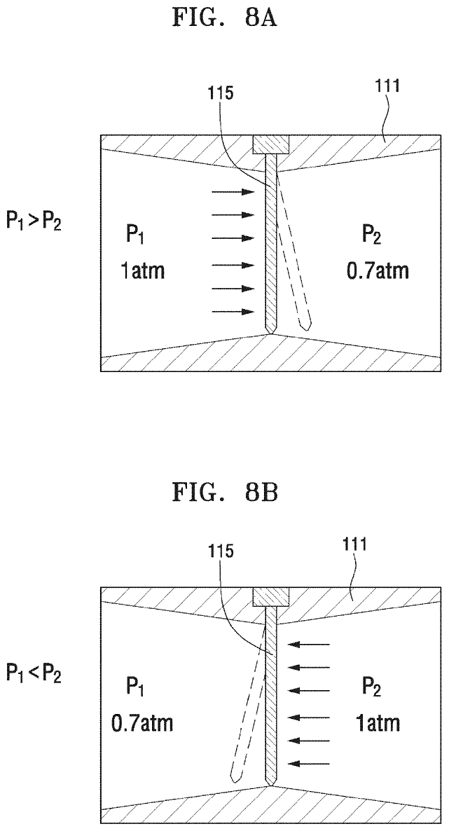

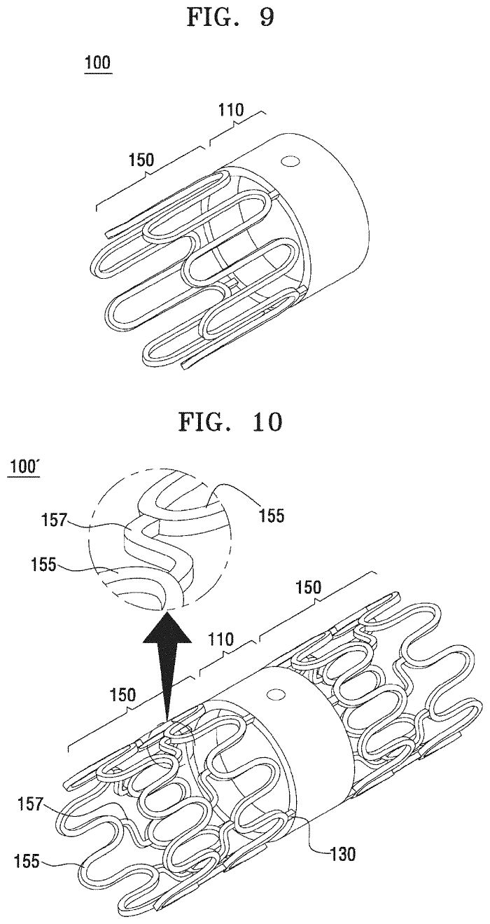

FIG. 2 is a perspective view of a stent according to an embodiment of the present disclosure, FIG. 3 is a perspective view showing a shape-memory state of FIG. 2, FIG. 4 is a perspective view of a pressure controller according to an embodiment of the present disclosure, FIG. 5 is a cross-sectional view of FIG. 4, FIG. 6 is a front view of a rotating portion of FIG. 5, FIG. 7 is a side view of FIG. 6, FIGS. 8A and 8B are diagrams illustrating operations of the pressure controller according to a pressure change in a eustachian tube, and FIG. 9 is a perspective view of a stent of FIG. 2 according to a modified example.

Frist, a stent 100 to be inserted in a eustachian tube for treatment of eustachian tube dysfunction according to an embodiment of the present disclosure may include a pressure controller 110 and a eustachian tube expansion portion 150. The eustachian tube expansion portion 150 expands the eustachian tube when being inserted in the eustachian tube. Here, the eustachian tube expansion portion 150 is entirely formed as a cylindrical shape having a hollow portion penetrating therein. Therefore, a passage through which the air and secreta from a middle ear move may be formed in the eustachian tube via the hollow portion.

As shown in FIG. 2, the eustachian tube expansion portions 150 are connected respectively to opposite ends of the pressure controller 110 that will be described later.

Here, the eustachian tube expansion portion 150 according to the embodiment has a shape that is transformed in a radial direction of the eustachian tube due to elasticity, and thus, the eustachian tube expansion portion 150 may be widened in the radial direction. Therefore, because the eustachian tube expansion portion 150 is widened and enlarged in the radial direction when the stent 100 is inserted in the eustachian tube, the eustachian tube may be effectively expanded, and moreover, the stent 100 may be stably fixed in the eustachian tube.

The shape of the eustachian tube expansion portion 150 will be described in detail below. As shown in FIG. 2, a metal wire extends in the back and forth directions of the eustachian tube in a pattern in which a waveform is repeatedly arranged, such that a ring shape may be formed in a circumferential direction of the eustachian tube. Therefore, an outer side of a ring surface formed by the waveform is in contact with the eustachian tube, and inside of the ring surface forms a hollow portion through which a fluid moves.

As described above, due to the shape characteristics of the pattern in which a certain type of waveform is repeated, the eustachian tube expansion portion 150 according to the present disclosure may be easily widened in the radial direction of the eustachian tube. That is, when valleys of adjacent waveforms and peaks of adjacent waveforms are close to each other or away from each other, the entire shape of the eustachian tube expansion portion 150 may be enlarged or contracted in the radial direction of the eustachian tube. In more detail, one side of the eustachian tube expansion portion 150 is connected to one side of the pressure controller 110 via connection frames 130 of short wire types as shown in FIG. 2. Here, a plurality of connection frames 130 may be formed in the circumferential direction. A part in one side of the eustachian tube expansion portion 150, which is connected to the pressure controller 110 via the connection frames 130, may not be easily transformed as compared with other parts. Thus, the eustachian tube expansion portion 150 is transformed in the radial direction, but may be transformed totally as a trumpet, that is, another end portion opposite to the connection frames 130 is further widened as shown in FIG. 3.

FIG. 3 shows a state in which the stent 100 of FIG. 2 is inserted in the eustachian tube and then the eustachian tube expansion portion 150 is expanded in the radial direction due to the elasticity. Therefore, the eustachian tube expansion portion 150 that is contracted as shown in FIG. 2 is mounted in a certain device for inserting the stent 100 in the eustachian tube, and then the stent 100 is located in the eustachian tube. In addition, the device is removed, the eustachian tube expansion portion 150 is widened in the radial direction due to the elastic transformation to the state shown in FIG. 3, and then expands the eustachian tube and is fixed in the eustachian tube.

Here, the waveform shown in FIG. 2 is formed such that the intermediate portion extends straightly and the end portion is extended as an arc connecting two adjacent straight wires to form the ring surface, but the waveform forming the ring surface is not limited thereto and may be variously transformed. For example, a straight line may extend in zig-zags.

In particular, the pressure controller 110 as described later and the eustachian tube expansion portion 150 determine a length of the stent 100 in the lengthwise direction, and the eustachian tube expansion portion 150 may be longer than the pressure controller 110. In addition, the stent 100 may have a sufficient length in the lengthwise direction. When the length of the stent 100 in the lengthwise direction is too short as compared with the length of the eustachian tube, the eustachian tube may not be widened such that opposite ends thereof are opened due to the stent 100, but only the intermediate portion of the eustachian tube is widened, even when the eustachian tube is widened by using the eustachian tube expansion portion 150. Therefore, the length of the stent 100 including the eustachian tube expansion portion 150 in the lengthwise direction may vary depending on a degree of blocking the eustachian tube, the length of the eustachian tube, etc.

Here, the eustachian tube expansion portion 150 according to the present disclosure may include a shape memory alloy material such that the shape transformation in the radial direction may be facilitated. That is, as shown in FIG. 3, the shape of widening in the radial direction of the eustachian tube in the eustachian tube (in more detail, the shape of widening as a trumpet shape) is thermally processed to be memorized. Thus, after the shape of FIG. 2 is inserted in the eustachian tube, the transformation into the shape of FIG. 3 may be easily executed. In the present embodiment, nitinol (Ni—Ti) is used as the shape memory alloy, but the shape memory alloy is not limited thereto.

Moreover, a biocompatible material that prevents bacterial proliferation is coated on the surface of the eustachian tube expansion portion 150, and thus, side effects that may occur after inserting the stent 100 in the eustachian tube may be minimized. Here, the biocompatible material may be coated by forming a titanium coating layer on the surface of the eustachian tube expansion portion 150, and then forming a titanium dioxide coating layer on the titanium coating layer. The titanium dioxide coating layer has an antibacterial effect for suppressing bacteria (biofilm).

Moreover, a biodegradable polymer layer such as PLGA is coated on the surface of the eustachian tube expansion portion 150, and drugs such as inflammation inhibitors, antibiotics, etc. are absorbed by the biodegradable polymer layer such that the drug may be slowly discharged into the eustachian tube through the biodegradable polymer layer. Here, the biodegradable polymer layer may be coated on the coating layer including the biocompatible material.

The structure of discharging the drug by coating the biocompatible material or coating the biodegradable polymer layer may be also applied to the pressure controller 110 that will be described later.

The tubal stenosis may be fundamentally addressed by expanding the eustachian tube due to the eustachian tube expansion portion 150. However, the eustachian tube has to be closed as necessary, the pressure controller 110 allows the pressure in the tympanic cavity and the pressure of outside to be adjusted via opening/closing of the eustachian tube.

The pressure controller 110 connected to one side of the eustachian tube expansion portion 150 blocks the eustachian tube, and opens/closes the eustachian tube according to a pressure difference between front and back portions thereof to control the pressure in the eustachian tube.

As shown in FIGS. 4 and 5, the pressure controller 110 may include a fixed portion 111 and a rotating portion 115.

The fixed portion 111 is formed as a cylindrical shape and has a hollow portion therethrough. Therefore, an outer surface of the fixed portion 111 is in contact with an inner surface of the eustachian tube, so as to support the inserted state of the pressure controller 110 in the eustachian tube. Here, opposite ends of the fixed portion fix the eustachian tube expansion portions 150 via the connection frames 130.

The rotating portion 115 is plate-shaped and shields the hollow portion in the fixed portion 111. Because the hollow portion is formed to be circular, the rotating portion 115 may be also formed as a disc shape.

Here, only an end portion on a side surface of the rotating portion 115 in the radial direction is fixed to the inner surface of the fixed portion 111, and thus, the rotating portion 115 may be rotated in the back and forth directions according to the pressure difference between front and rear portions of the rotating portion 115. Therefore, the front and rear portions of the rotating portion 115 may be shielded or opened according to the rotation of the rotating portion 115.

The rotating portion 115 may include a shielding portion 116 that is a disc-shaped member to cover the hollow portion surface in the fixed portion 111, and a protruding coupling portion 117 that protrudes from the end portion on the side surface of the shielding portion 116 in the radial direction, as shown in FIGS. 6 and 7.

A coupling groove portion (not shown) in which the protruding coupling portion 117 is inserted may be formed in a side of the inner surface of the fixed portion 111. Although not shown in the drawings, the coupling groove portion is formed to extend from a certain location of the inner surface of the fixed portion 111 to an end of an opened surface of the fixed portion 111, and then, the coupling groove portion moves the protruding coupling portion 117 into the fixed portion 111 while the protruding coupling portion 117 is inserted therein at the end of the opened surface such that the rotating portion 115 is positioned in the fixed portion 111. Then, a space between the protruding coupling portion 117 and the coupling groove portion is bonded by the laser to rotatably fixes the rotating portion 115.

In FIGS. 8A and 8B, based on the pressure controller 110, when it is assumed that a left side P₁ is connected to the middle ear and a right side P₂ is connected to the nasal cavity, the rotating portion 115 rotates to the right side due to the pressure difference when the pressure in the middle ear is greater than that of the outside, and then, opens the eustachian tube such that the pressures of P₁ and P₂ are equal to each other. When the pressure of the outside is greater than that of the middle ear, the rotating portion 115 rotates to the left side due to the pressure difference and opens the eustachian tube such that the pressures of P₁ and P₂ are equal to each other. Here, the rotating portion 115 may be manufactured to rotate when the pressure difference between the front and rear portions of the rotating portion 115 is about 0.3 atm.

As shown in the enlarged view of FIG. 7, the side surface of the shielding portion 116 opposite to the protruding coupling portion 117 in the radial direction may have an inclined surface that is inclined toward the front or rear surface. Therefore, the side surface includes a horizontal contact surface that is in contact with the fixed portion 111, along with the inclined surface. Due to the inclined surface, interference due to the contact may be reduced when the rotating portion 115 rotates.

FIG. 9 shows a modified example of the stent 100 described above with reference to FIGS. 2 and 8, and as shown in FIG. 9, the eustachian tube expansion portion 150 may be only formed on one side of the pressure controller 110. That is, in the above-described embodiment, the eustachian tube expansion portions 150 are symmetrically formed on opposite sides of the pressure controller 110, but in the present modified example, the eustachian tube expansion portion 150 is only formed on one side of the pressure controller 110.

Also, the eustachian tube expansion portion 150 may be positioned at the side of the middle ear or at the side of the nasal cavity when the stent 100 is inserted in the eustachian tube.

Next, the stent 100 according to another embodiments of the present disclosure will be described below with reference to FIGS. 10 and 11.

In the embodiment described below, differences from the above-described embodiments will be described. The pressure controller 110 is the same as that of the above embodiment. FIGS. 10 and 11 show that the eustachian tube expansion portions 150 are formed on opposite sides of the pressure controller 110, but the eustachian tube expansion portion 150 may be formed only on one side as shown in FIG. 9.

In the embodiment of FIG. 10, a plurality of body portions 155 that each form a ring shape in the circumferential direction as a pattern in which a waveform of a wire is repeated in the back and forth direction of the eustachian tube as described in the above embodiment are formed in the lengthwise direction in the eustachian tube, and the body portions 155 are connected to each other via a connection portion 157 therebetween. Here, the connection portion 157 is also formed as a wire.

Here, the connection portion 157 may be formed straight or curved, for example, may have ‘S’-shape as shown in the drawings. The connection portion 157 of the ‘S’-shape may have elasticity, and may connect the adjacent body portions 155 such that more effective elastic transformation may be executed.

A position where a stent 100′ is inserted in the eustachian tube may not be straight. Thus, due to the shape characteristic in which the plurality of body portions 155 are connected via the connection portion 157, the stent 100′ may be effectively curved in the curved eustachian tube. At the same time, the transformation due to the elasticity in the radial direction of the eustachian tube may be easily performed as described above. Here, like in the above-described embodiment, the shape of widening as the trumpet due to the connection to the pressure controller 110 may be memorized.

Here, as in the above embodiment, the stent 100′ according to the present embodiment may be also formed of a shape memory alloy, the biocompatible material may be coated to reduce side effects, and the biodegradable polymer layer may be coated to discharge the drug for treating the eustachian tube or prevention drug.

In the embodiment shown in FIG. 11, body portions 165 each having a ring shape, in which a waveform extends in a circumferential direction while repeating in the circumferential direction of the eustachian tube, are formed, and a plurality of body portions 165 are arranged in the back and forth directions of the eustachian tube to be spaced apart from one another. Here, the body portions 165 spaced apart from one another are connected via connecting portions 167.

Here, a plurality of connecting portions 167 may be formed as straight lines each connecting peaks of the waveforms, which form the outermost part of the body portions 165. Therefore, when a stent 100″ is inserted in the eustachian tube, the connecting portions 167 of the eustachian tube expansion portion 150 and the peaks of the body portions 165 are in contact with the inner wall of the eustachian tube, and accordingly, it may be easy to transform the shape in the radial direction of the eustachian tube due to the shape characteristic of the body portions 165, each of which is formed as the waveform. Here, as described above, the shape of widening as the trumpet due to the connection to the pressure controller 110 may be memorized.

Also, although not shown in the drawing, the connecting portion 167 may connect a single body portion 165 located at the end portion of the eustachian tube expansion portion 150 and the pressure controller 110 to each other may be provided.

Here, as in the above embodiment, the stent 100″ according to the present embodiment may be also formed of a shape memory alloy, the biocompatible material may be coated to reduce side effects, and the biodegradable polymer layer may be coated to discharge the drug for treating the eustachian tube or prevention drug.

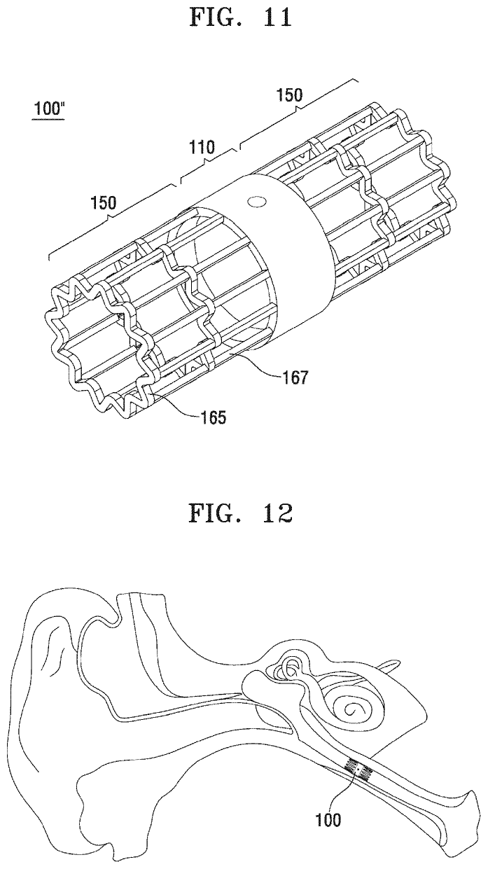

FIG. 12 is a diagram showing an example of an insertion procedure of the stent 100, 100′, 100″ in the eustachian tube for treating eustachian tube dysfunction according to the present disclosure.

As shown in FIG. 12, when the stent 100 according to the present disclosure is inserted in the eustachian tube, the eustachian tube is expanded by the eustachian tube expansion portion 150 and the stent 100 may be fixed, and a pressure difference between the tympanic cavity and the outside may be adjusted by the pressure controller 110 located at one side of the eustachian tube expansion portion 150. Therefore, the eustachian tube stenosis and the abnormal patency of the eustachian tube may be simultaneously treated by using the stent 100 according to the present disclosure.

The stent 100 according to the present disclosure may be easily and simply inserted through a nose hole and does not make problems even when being inserted permanently.

It is to be understood that the present disclosure is not limited to the disclosed embodiments, but, on the contrary, may be embodied in various embodiments. Various modifications may be made to the disclosure by one of skilled in the art without departing from the spirit and scope of the disclosure as defiled in the appended claims.

1. A stent to be inserted in an eustachian tube for treatment of eustachian tube dysfunction, the stent comprising:
a pressure controller configured to block the eustachian tube and be opened/closed according to a pressure difference between front and rear portions thereof to control a pressure in the eustachian tube; and
a eustachian tube expansion portion being connected to the pressure controller and having a hollow portion passed therein to make a fluid move in back and forth directions of the eustachian tube, the eustachian tube expansion portion configured to be inserted in the eustachian tube to expand the eustachian tube by transforming a shape thereof in a radial direction of the eustachian tube.

2. The stent of claim 1, wherein the eustachian tube expansion portion is connected to one side or opposite sides of the pressure controller.

3. The stent of claim 1, wherein the eustachian tube expansion portion includes a body portion which forms a ring shape while extending in a circumferential direction as a pattern in which a waveform of a wire is repeated in the back and forth directions of the eustachian tube.

4. The stent of claim 3, further comprising a plurality of connecting frames connecting one side of the eustachian tube expansion portion to one side of the pressure controller.

5. The stent of claim 3, wherein a plurality of the body portions are arranged in the back and forth directions of the eustachian tube, and
wherein the eustachian tube expansion portion further includes a connecting portion connecting the body portions.

6. The stent of claim 5, wherein the connecting portion is formed as a wire.

7. The stent of claim 5, wherein the connector is curved as an S-shape.

8. The stent of claim 1, wherein the eustachian tube expansion portion includes:
a body portion which forms a ring shape while extending in a circumferential direction of the eustachian tube as a pattern in which a waveform is repeated in the circumferential direction, a plurality of the body portions being arranged in the back and forth directions of the eustachian tube; and
a connecting portion connecting the body portions or connecting the body portion and the pressure controller.

9. The stent of claim 1, wherein the eustachian tube expansion portion is formed of a shape memory alloy and thermally treated to memorize a shape that is transformed in the radial direction of the eustachian tube.

10. The stent of claim 9, wherein the eustachian tube expansion portion is formed of nitinol (Ni—Ti).

11. The stent of claim 1, further comprising a biocompatible material coated on a surface of the stent and configured to prevent bacterial proliferation.

12. The stent of claim 1, further comprising a biodegradable polymer layer coated on a surface of the stent and configured to absorb and discharge a drug into the eustachian tube therethrough.

13. The stent of claim 1, wherein the pressure controller comprises:
a fixed portion configured to be inserted and support the eustachian tube, the fixed portion being formed as a cylindrical shape and having a hollow portion therethrough; and
a rotating portion shielding a surface of the hollow portion in the fixed portion, an end portion of a side surface in a radial direction of the rotating portion being fixed to an inner surface thereof, and the rotating portion rotating due to a pressure difference between front and rear portions thereof to open/close the surface of the hollow portion.

14. The stent of claim 13, wherein the rotating portion includes:
a shielding portion covering the surface of the hollow portion in the fixed portion; and
a protruding coupling portion protruding from an end portion of a side surface in a radial direction of the shielding portion, and
a coupling groove portion in which the protruding coupling portion is inserted is formed in the inner surface of the fixed portion.

15. The stent of claim 14, wherein, a side surface of the shielding portion opposite to the protruding coupling portion in a radial direction has an inclined surface that is inclined toward a front surface or a rear surface, and forms a contact surface that is in contact with the inclined surface and the fixed portion.
